(19) United States
(12) Patent Application Publication
(10) Pub. No.: US 2024/0226892 A9
(43) Pub. Date: Jul. 11, 2024

(54) RAPID, MICROFLUIDIC DIAGNOSTIC DEVICE AND METHOD FOR BIOLOGICAL SEX DETERMINATION

(71) Applicant: Definitive Biotechnologies LLC, Elkridge, MD (US)

(72) Inventors: Timothy Alcorn, Raleigh, NC (US); Peter Latham, Acton, MA (US)

(21) Appl. No.: 18/493,412

(22) Filed: Oct. 24, 2023

Related U.S. Application Data
(60) Provisional application No. 63/380,715, filed on Oct. 24, 2022.

(51) Int. Cl. B01L 3/00

(52) U.S. Cl. CPC B01L 3/502761; B01L 3/502715; B01L 2300/048; B01L 2400/0605

(57) ABSTRACT

A device where saliva from a pregnant woman in the first trimester of pregnancy (6-8 weeks of pregnancy) is mixed with a chaotropic agent that facilitates cell lysis and protein denaturization. The mixture is introduced into a microfluidic system where the mixture is passed across a solid-state structure that captures all DNA in the mixture including cfDNA. After removal of protein and other waste from the membrane, the captured DNA is eluted from the solid-state structure into nucleic acid amplification reaction chambers. The system contains individual chambers for a positive control chamber and the target, male-specific, Y chromosome nucleotide sequence. The presence of amplification products is then detected in such a manner that a lay user can accurately determine the presence or absence of the Y chromosome. The presence of a Y chromosome is indicative of a biologically male fetus.

### CROSS-REFERENCE TO RELATED PATENT APPLICATION

This patent application claims priority under 35 U.S.C. § 119(e) to U.S. provisional patent application No. 63/380,715, filed Oct. 24, 2022, entitled “RAPID, MICROFLUIDIC DIAGNOSTIC DEVICE AND METHOD FOR BIOLOGICAL SEX DETERMINATION,” which is hereby incorporated by reference in its entirety as part of the present disclosure.

### FIELD OF THE INVENTION

The present invention relates to devices and methods for determination of the biological sex of a fetus based on the detection presence of circulating free fetal chromosomal DNA (cfDNA) in minimally invasive maternal fluids such as saliva or peripheral blood at the point-of-need (PON).

### BACKGROUND INFORMATION

Biological sex determination in early pregnancy is most typically done via ultrasound in the second trimester of pregnancy (>14 weeks of pregnancy). Alternatively, analysis of free fetal chromosomal DNA (cfDNA) or cell-free fetal DNA (cffDNA) (collectively referred to herein as “cfDNA”) can be used to determine fetal gender as early as 6-8 weeks of pregnancy by analyzing the presence or absence of the gender specific Y chromosome. However, this method typically requires a venous blood drawn and shipped to a central laboratory where molecular technologies such as PCR can be performed, all requiring trained professionals. Peripheral blood can be used as an alternative to venous blood, thus allowing for collection by the lay person. However, peripheral blood requires sample preparation to remove red blood cells which inhibits molecular technologies such as PCR before processing. Therefore, this sample type also requires testing by a trained professionals at a remote, centralized laboratory.

Saliva is a minimally invasive sample type and as such is an attractive sample for use at the PON since it does not require a professional for collection as it does not require a venous puncture or a complicated device for sample collection and yet contains most of the biomarkers found in blood including cfDNA. However, unless saliva is incorporated for use with a device suitable for operation by the lay person, saliva samples still require shipping to a centralized remote laboratory where professionals can conduct testing. Such a PON device would need to be able to deal with viscosity and bubbles inherent in saliva, which can affect fluid transport, be able to efficiently isolate and detect chromosomally cfDNA, and facilitate interpretation without the need for a professional.

It is an object of the present invention, and/or of the currently preferred embodiments thereof, to overcome one or more of the above-described drawbacks and/or disadvantages of the prior art.

### SUMMARY OF THE INVENTION

In accordance with a first aspect, the present invention is directed to a device which incorporates a methodology for collection of maternal fluid, such as saliva, isolation of cfDNA therein using a solid-state membrane, removal or substantial removal of any materials in the saliva or other maternal fluid that could adversely affect the performance of the device, amplification of the isolated chromosomal cfDNA, and a visualized result and interpretation.

In some embodiments of the present invention, the device is configured, and the method is directed to collecting maternal fluid, such as saliva, from a pregnant woman in the first trimester of pregnancy (6-8 weeks of pregnancy) and mixing the collected saliva or other maternal fluid with a chaotropic agent that facilitates cell lysis and protein denaturization. The mixture is introduced into a microfluidic system where the mixture is passed across a solid-state structure that captures DNA, or substantially all DNA in the mixture, including cfDNA. After removal of protein(s) and other waste from the membrane, the captured DNA is eluted from the solid-state structure into nucleic acid amplification reaction chambers. The system contains at least one reaction chamber including a positive control reaction chamber or chamber portion and another chamber or chamber portion for the target, male-specific, Y chromosome nucleotide sequence. The presence of amplification products is then detected in such a manner that the lay user can accurately determine the presence or absence of the Y chromosome. The presence of a Y chromosome is indicative of a biologically male fetus.

In some embodiments of the present invention, the device comprises a sample port or chamber for receiving therein a sample-containing mixture containing therein a biological sample, such as saliva or other maternal fluid from a pregnant woman, as summarized above. A solid-state membrane is in fluid communication with the sample port or chamber and is configured to receive the sample-containing mixture therefrom, and allow the sample-containing mixture to pass across the membrane and capture nucleic acids, including fetal chromosomal cfDNA, in the biological sample on the membrane. A first pump is in fluid communication with at least one of the solid-state membrane or a waste chamber. Actuation of the first pump causes the sample-containing mixture to flow across the solid-state membrane and into the waste chamber. The device further comprises an eluent chamber containing an eluent therein, an eluent reservoir in fluid communication with the solid-state membrane, and a second pump in fluid communication with at least one of the solid-state membrane or the eluent chamber. Actuation of the second pump causes the eluent to flow from the eluent chamber across the solid-state membrane, elute captured nucleic acids from the solid-state membrane, including fetal chromosomal cfDNA, and flow with the captured nucleic acids into the eluent reservoir where the eluent and captured nucleic acids are pre-mixed in the eluent reservoir. In a currently preferred embodiment, a plurality of reaction chambers are in fluid communication with the eluent reservoir for receiving therefrom the eluent and captured nucleic acids. A first reaction chamber is configured for a positive control, and a second reaction chamber is configured for the detection of Y chromosomal DNA in the fetal chromosomal cfDNA.

In some embodiments of the present invention, the first pump is a syringe containing a barrel and a plunger received within the barrel. The barrel defines the waste chamber therein. Movement of the plunger either draws or pulls, or pushes the saliva or other maternal-fluid containing sample mixture across the solid-state membrane and into the waste chamber of the barrel.

In some embodiments of the present invention, the second pump is movable between a non-actuated position and an actuated position. The eluent chamber includes a frangible or breakable wall that is breakable by movement of the second pump between the non-actuated position and the actuated position to pump eluent from the eluent chamber across the solid-state membrane and into the eluent reservoir. In some such embodiments, the second pump is a plunger. Movement of the plunger from the non-actuated position to the actuated position breaks the frangible or breakable wall of the eluent chamber and pushes or pulls the eluent across the solid-state membrane and into the eluent reservoir. In other embodiments, the second pump is a syringe including a barrel and a plunger received within the barrel. The barrel defines the eluent chamber therein, and movement of the plunger pushes or pulls the eluent across the solid-state membrane and into the eluent reservoir.

Some embodiments of the present invention further comprise a first one-way valve in fluid communication between the sample port or chamber and the solid-state membrane and configured to allow the sample-containing mixture to flow in the direction from the sample port or chamber to the solid-state membrane, but prevent liquid flow in the opposite direction.

Some embodiments of the present invention further comprise a second one-way valve in fluid communication between the solid-state member and the eluent reservoir and configured to allow fluid flow in the direction from the solid-state member into the eluent reservoir, but prevent liquid flow in the opposite direction.

Some embodiments of the present invention further comprise at least one capillary conduit in fluid communication between the eluent reservoir and at least one reaction chamber. The capillary conduit(s) is (are) configured to allow the eluent with captured nucleic acids to flow by capillary action through the capillary conduit(s) and into the reaction chamber(s).

In accordance with another aspect, the present invention is directed to a device comprising: (i) first means for receiving therein a sample-containing mixture containing therein a biological sample from a pregnant woman; (ii) second means in fluid communication with the first means for receiving the sample-containing mixture therefrom, for allowing the sample-containing mixture to pass across the second means, and for capturing fetal chromosomal cfDNA in the biological sample on the membrane; (iii) third means for receiving and holding the sample-containing mixture after passing across the second means; (iv) fourth means in fluid communication with at least one of the second means or the third means for pumping the sample-containing mixture across the second means and into the third means; (v) fifth means for containing an eluent therein and for allowing the eluent to flow across the second means after the sample-containing mixture passes across the second means, and for removing from the second means fetal chromosomal cfDNA from the biological sample with the eluent; (vi) sixth means in fluid communication with the second means for receiving and collecting the eluent with captured fetal chromosomal cfDNA from the biological sample therein; (vii) seventh means in fluid communication with at least one of the second means or the fifth means for pumping the eluent from the fifth means across the second means and eluting fetal chromosomal cfDNA from the second means into the sixth means; and (viii) eighth means in fluid communication with the sixth means for receiving therefrom the eluent and captured fetal chromosomal cfDNA and for detecting Y chromosomal DNA, if any, in the fetal chromosomal cfDNA.

In some embodiments of the present invention, the first means is a sample port or chamber, the second means is a solid-state membrane, the third means is a waste chamber, the fourth means is a pump, the fifth means is an eluent chamber, the sixth means is an eluent reservoir, the seventh means is a pump, and the eighth is a reaction chamber configured for a positive control, and another reaction chamber configured for the detection of Y chromosomal DNA, if any, in the fetal chromosomal cfDNA.

In accordance with another aspect, the present invention is directed to a formulation for collecting a biological sample of maternal fluid, such as saliva, and capturing nucleic acids, including chromosomal cfDNA and a Y chromosome nucleotide sequence, if any, in the collected biological sample on a solid-state membrane. The formulation comprises: (i) one or more non-toxic chaotropic agents; (ii) ethanol; and (iii) coloring and/or flavoring agents. The formulation is receivable within a cavity of a pregnant woman, such an oral cavity or nasal cavity, to collect the biological sample of maternal fluid therefrom. The one or more non-toxic chaotropic agents lyse the cells of the biological sample, if necessary, and the ethanol binds chromosomal cfDNA, and a Y chromosome nucleotide sequence, if any, in the lysed cells of the biological sample to the solid-state membrane.

Some embodiments of the present invention comprise about 0.1% to about 40% w/v non-toxic chaotropic agents and about 5% to about 30% w/v ethanol. In some such embodiments, the non-toxic chaotropic agents are selected from the group including the following individually or in any combination thereof: (i) about 5% to about 30% w/v urea; about 0.1% to about 3% w/v sodium lauryl sulfate; and about 2% to about 40% w/v ammonium trichloroacetate.

In some embodiments of the present invention, the formulation is provided in combination with a long-chain fatty alcohol wash configured to flow over the solid-state membrane following the formulation to substantially eliminate any residual ethanol of the formulation on the solid-state membrane.

One advantage of the present invention, and/or of embodiments thereof, is that it can provide a solution to the saliva collection issues encountered in the above-described prior art. Another advantage is that the system allows the use of a mouthwash or nasal wash that a user can swish in the mouth or spray into the nose before introduction of the saliva or nasal fluid into the microfluidic or other nucleic acid isolation and purification system. Yet another advantage is that the mouthwash or nasal wash contains sample preparation chemicals that are non-toxic thus allowing partial and/or complete sample preparation (e.g., cell lysis) before introduction into the microfluidic or other nucleic acid isolation and purification system or into an external sample collection cup or device. A further advantage of the invention and/or of embodiments thereof, is that they can normalize the volume of sample collected, eliminate or substantially eliminate issues of viscosity and foaming, and eliminate, substantially eliminate or minimize the exposure of users to toxic chemicals. Yet another advantage is that the invention can be extended to a nasal wash for easier or different collection of nasal samples.

A still further advantage of the invention and/or of embodiments thereof is that they enable determination of the biological sex of a fetus based on the detection presence of circulating free fetal chromosomal cfDNA in minimally invasive maternal fluids, such as saliva, nasal fluid or peripheral blood, at the point of need.

Other objects and advantages of the present invention, and/or of embodiments thereof, will become more readily apparent in view of the following detailed description of embodiments and accompanying drawings.

### BRIEF DESCRIPTION OF THE DRAWINGS

FIG. 1A is a somewhat schematic illustration of an embodiment of a microfluidic system of the present invention indicated generally by the reference number 10 comprising a microfluidic device 12 for receiving through an inlet port 14 a sample mixture 16. The sample mixture 16 contains a biological sample, such as saliva or nasal fluid (e.g., mucus), non-toxic chaotropic agents for cell lysis, ethanol and coloring and/or flavoring agents. The sample mixture 16 is introduced into the inlet port 14 of the microfluidic device 12 by a transfer device, such as from a syringe 18A, funnel 18B or collection vessel 18C, and through a sample introduction tube 18D. The inlet port 14 is in fluid communication with a capture membrane 20 (e.g., a glass or solid-state membrane), and a syringe or other pump 22 is actuated to draw or pull the sample mixture 16 across or through the solid-state membrane 20 where cfDNA in the sample is captured by and collected on the membrane and the remainder of the sample mixture is pulled into the syringe 22 as waste. An elution blister or other pump 24 containing eluent 26 (e.g., water (lined with the color blue)) is then depressed or actuated to break the blister and release and pump the eluent across the solid-state membrane 20 to remove the captured cfDNA on the membrane and carry the captured cfDNA into an eluent reservoir 28 where the eluent with captured cfDNA is allowed to collect or pool. A plurality of capillary tubes or conduits 30, 30 are connected in fluid communication between the reservoir 28 and a plurality of reaction chambers 32, 32, and are configured to transfer the eluent with captured cfDNA by capillary action from the reservoir into the reaction chambers which include a positive control reaction chamber, and a reaction chamber for the amplification and detection of fetal chromosomal cfDNA.

FIG. 1B are somewhat schematic illustrations of the syringe 18A, funnel 18B and sample collection vessel 18C of FIG. 1A, each connected or connectable in fluid communication with the sample introduction tube 18D preferably by a lockable or locking connector 50.

FIG. 2 is a somewhat schematic illustration of another embodiment of the microfluidic system 10 of FIG. 1 where the microfluidic device 12 includes an elution syringe or other pump 24′ prefilled with eluent 26 (e.g., water (lined for the color blue)) instead of the elution blister 24 of FIG. 1. The elution syringe 24′ is actuated to release and pump the eluent 26 across the solid-state membrane 20 to remove the captured cfDNA on the membrane and carry the captured cfDNA into the eluent reservoir 28 where the eluent 26 with captured cfDNA is allowed to collect or pool prior to transfer by capillary action into the reaction chambers 32, 32.

FIG. 3 is a somewhat schematic illustration of the microfluidic system 10 of FIG. 1 showing a first step of use of the device 12 (which step also applies to use of the device of FIG. 2) where a user collects a biological sample and introduces a sample mixture 16 into the microfluidic device in accordance with any of three options 18A, 18B or 18C.

FIG. 4 is a somewhat schematic illustration of the microfluidic system 10 of FIG. 1 showing a second step of use of the device 12 (which step also applies to use of the device of FIG. 2) where a patient or other user pulls the empty syringe 22 of the microfluidic device 12 to, in turn, pull the sample mixture 16 (lined for the color green) across the capture membrane 20 and into the barrel of the syringe 22 which acts as waste container.

FIG. 5 is a somewhat schematic illustration of the microfluidic system 10 of FIG. 1 showing a third step of use of the device 12 (which step also applies to use of the device of FIG. 2) where the patient or other user continues to pull the plunger of the syringe 22 of the device 12 which either pulls dodecanol (or other long-chain fatty alcohol) 36 (lined for the color red) across the membrane 20 and/or air across the membrane 20 to remove substantially all residual ethanol from the membrane. In one embodiment, there is no need for dodecanol and residual air in the syringe, funnel, collection cup or other transfer device eliminates or substantially eliminates the dodecanol

FIG. 6A is a somewhat schematic illustration of the microfluidic system 10 of FIG. 1 showing a fourth step of use of the device 12 (which step also applies to use of the device of FIG. 2) where a patient or other user depresses the elution blister 24 which pushes the elution buffer 26 across the membrane 20 to elute the captured DNA and RNA (e.g., cfDNA) off the membrane 20 and into the capture reservoir 28 (where the eluent with captured cfDNA is lined for the color blue). Fluid flow is controlled by one-way valves 38, 38 (shown as arrows lined for the color red) (i) in fluid communication between the inlet port 14 and the inlet side of the membrane 20, and (ii) in fluid communication between the outlet side of the membrane 20 and the capture reservoir 28. The valves 38, 38 prevent liquid from flowing backwards from the capture reservoir 28 and/or reaction chambers 32, 32 into the membrane 20, and prevent liquid and/or other material from flowing backwards through the inlet port 14 and into the transfer/collection device(s) 18A, 18B, 18C and/or 18D. Once the captured RNA and DNA (e.g., cfDNA) is in the capture reservoir 28, the reaction chambers 32, 32 are filled via capillary action by the capillary conduits 30, 30 extending in fluid communication between the capture reservoir 28 and each respective reaction chamber 32, 32, where the eluent dissolves the reaction chemistry pre-stored in the reaction chambers as lyophilized beads (circles lined for the color yellow). Depression of the elution blister 24 also activates a device heating element 42 in thermal communication with the reaction chambers 32, 32 for heating the chambers to the required temperature for the reaction.

FIG. 6B is cross-sectional view of the syringe barrel of the device of FIG. 6A showing the collected waste dodecanol 36 (lined for the color red) on top of the waste sample mixture 16 (lined for the color green) within the barrel of the syringe 22.

FIG. 7 is a somewhat schematic illustration of the microfluidic system 10 of FIG. 2 showing a fourth step of use of the device 12 (which step also applies to use of the device of FIG. 2) where a patient or other user depresses or otherwise actuates the elution syringe 24′ which pushes the elution buffer 26 across the membrane 20 to elute the captured DNA and RNA (e.g., cfDNA) off the membrane and into the capture reservoir 28 (where the elution buffer with captured DNA and RNA (e.g., cfDNA) is lined for the color blue). Fluid flow is controlled by the one-way valves 38, 38 (shown as arrows lined for the color red) (i) in fluid communication between the inlet port 14 and the inlet side of the membrane 20, and (ii) in fluid communication between the outlet side of the membrane 20 and the capture reservoir 28. The valves 38, 38 prevent fluid from flowing backwards from the capture reservoir 28 and/or reaction chambers 32, 32 into the membrane 20, and prevent any material from flowing backwards through the inlet port 14 into the transfer/collection device(s) 18A, 18B, 18C and/or 18D. Once the RNA and DNA (e.g., cfDNA) is in the capture reservoir 28, the reaction chambers 32, 32 are filled via capillary action through the capillary conduits 30, 30 which dissolves the reaction chemistry pre-stored in the chambers as lyophilized beads (circles lined for the color yellow). In FIG. 7, the pre-stored reaction chemistry is shown as a circle (lined for the color yellow) within each empty reaction chamber 32, 32, whereas in FIGS. 8 and 9 below, the reaction chemistry is not shown because at that stage it is dissolved in the elution buffer with captured DNA and RNA (e.g., cfDNA). Depression of the plunger of the elution syringe 24′ also activates the device 10 heating element 42 for heating the reaction chambers 32, 32 to the required temperature for the reaction. The reaction chambers 32, 32 are heated by the heating element 42 to initiate an amplification reaction where the target cfDNA is amplified, if present. Positive amplification is detected via user visualization, such as color change through a transparent/translucent window(s) 44, 44 of the reaction chamber(s) (e.g., from yellow to green, as shown), fluorescence, or a change in turbidity, or by digital image capture via an imbedded CCD camera or an external device such as a smart phone (not shown).

FIG. 8 is a somewhat schematic illustration of the microfluidic system 10 of FIG. 1 showing a fifth step of use of the device 12 (which step also applies to use of the device of FIG. 2) to identify a biological male fetus where the reaction chambers 32, 32 are heated by the heating element 42 of the device to initiate an amplification reaction where the target cfDNA is amplified, if present. Positive amplification is detected via user visualization, such as color change, through the transparent/translucent windows 44, 44 of the reaction chambers 32, 32. For example, as shown in FIG. 8, a positive amplification in each reaction chamber 32, 32 is lined for the color green (i.e., a color change from yellow to green) and is visible through the transparent/translucent windows 44, 44 of the reaction chambers 32, 32. As may be recognized by those of ordinary skill in the pertinent art based on the teachings herein, positive amplification may be detected in any of numerous different ways that are currently known, or that later become known, such as by fluorescence, a change in turbidity, or by digital image capture via an imbedded CCD camera or other sensor, or an external device such as a smart phone A Negative amplification reaction is indicated when there is no change in the color (or in the fluorescence, turbidity or digital image) from the start of the heating to the end of heating. A positive Y chromosome reaction (plus a positive control being positive) is indicative of a male fetus.

FIG. 9 is a somewhat schematic illustration of the microfluidic system 10 of FIG. 1 showing an alternative fifth step of use of the device 12 (which step also applies to use of the device of FIG. 2) to identify a biological female fetus where the reaction chambers 32, 32 are heated by the heating element 42 of the device 12 to initiate an amplification reaction where the target cfDNA chamber has a negative amplification reaction along with the positive amplification of the positive control. Positive amplification is detected via user visualization, such as color change through the transparent/translucent window(s) 44, 44 of the reaction chamber(s) 32, 32 (e.g., from yellow to green, as shown), fluorescence, or a change in turbidity, or by digital image capture via an imbedded CCD camera or an external device such as a smart phone (not shown). A Negative amplification reaction is indicated when there is no change in the color (or fluorescence, turbidity or digital image) from the start of the heating to the end of heating. A negative Y chromosome reaction (plus a positive control being positive) is indicative of a female fetus.

### DETAILED DESCRIPTION

In a currently preferred embodiment, the sample collection formulation is provided in the form of a mouthwash or a nasal spray or flush. The sample collection formulation 16 contains the following components:
1) Non-toxic chaotropic agents for cell lysis used individually or in combination. Such agents include but are not limited to urea which is used in artificial saliva; sodium lauryl sulfate which is used in toothpaste; ammonium trichloroacetate which is used to treat lesions on the skin and mucus membranes; and/or guanidinium chloride, guanidine hydrochloride, guanidinium thiocyanate and/or guanidinium isothiocyanate. The amount of urea is preferably within the range of about 5% to about 30% w/v, is more preferably within the range of about 10% to about 25% w/v, and is even more preferably within the range of about 15% to about 20% w/v. The amount of sodium lauryl sulfate is preferably within the range of about 0.1% to about 3% w/v, is more preferably within the range of about 0.2% to about 1.5% w/v, and is even more preferably within the range of about 0.4% to about 0.7% w/v. The amount of ammonium trichloroacetate is preferably within the range of about 2% to about 40% w/v, is more preferably within the range of about 4% to about 20% w/v, and is even more preferably within the range of about 8% to about 10% w/v. As may be recognized by those of ordinary skill in the pertinent art based on the teachings herein, some applications may not require cell lysis, such as where there is free-floating nucleic acids. However, such applications may nevertheless require the non-toxic chaotropic agents to chaotrope the cells and facilitate binding nucleic acids to the solid-state membrane or other nucleic acid capture device.
2) Ethanol to facilitate capture of RNA and DNA (e.g., cfDNA). The amount of ethanol is preferably within the range of about 5% to about 30% w/v, is more preferably within the range of about 10% to about 25% w/v, and is even more preferably within the range of about 15% to about 20% w/v.
3) Coloring and/or flavoring agents.

The following is a representative formulation:
1) about 20% w/v ethanol;
2) about 0.1% w/v sodium lauryl sulfate;
3) about 20% w/v ammonium trichloroacetate;
4) about 0.042% w/v menthol; and
5) water.

The device and method also can employ a long-chain fatty alcohol wash. Ethanol is an inhibitor of many amplification reactions. In the system disclosed herein, ethanol is used to bind nucleic acids to the glass or solid-state membrane 20 surfaces. Residual ethanol on the glass or solid-state membrane surface or in the fluidic channels can be carried into the reaction chambers 32, 32 when nucleic acids are eluted off the glass or solid-state membrane surfaces. To solve this issue, the fluidic channels and glass surfaces can be washed with a long chain (>4) fatty alcohol, such as 2-dodecanol, that is clear and hydrophobic. This provides several advantages: a) the fatty alcohol displaces and solubilizes residual ethanol in the system; b) residual fatty alcohol does not inhibit amplification reactions or resulting visualization; and c) residual fatty alcohol can be used to provide a barrier to evaporation of water from the reaction solutions. Other long chain fatty alcohols that can be used include but are not limited to the following (used individually or in any combination):
1) Dodecanol;
2) Octanol;
3) Stearyl Alcohol;
4) Lauryl Alcohol;
5) Cetyl Alcohol;
6) Oleyl Alcohol; and
7) Butyl Alcohol.

There are several options for using the above formulation as a mouthwash or nasal spray or wash, including the following:

Option 1: The mouthwash/nasal spray or wash is coated onto a gauze or other wad of absorbent material that is used either to swab the mouth or nose. The swab is then compressed by a syringe 18A in the inlet 14 of the microfluidic device 12 to release the materials therefrom and into the device. The syringe 18A may also contain 2-dodecanol 36 (about 50 to about 500 μl (or other long-chain fatty alcohol, as indicated above) for improved assay performance. The 2-dodecanol or other long-chain fatty alcohol(s) 36 sits in a chamber of the syringe 18A such that it is introduced into the device 12 following the saliva mixed with the mouthwash/nasal spray.

Option 2 (saliva only): The user swishes the mouthwash in the mouth and then spits the mouthwash with saliva into the microfluidic device with the help of a funnel 18B or like device.

Option 3 (saliva only): The user swishes the mouthwash in the mouth and then spits the mouthwash with saliva into a secondary collection vessel or cup 18C which is pre-loaded with additional chaotropic agents and/or ethanol 46 that are released before, upon or after sealing the cup. For example, the collection vessel 18C can include a chamber with a frangible or breakable wall containing therein the additional chaotropic agents and/or ethanol 46. The vessel closure may include a piercing member such that upon closing the vessel 18C with the closure, the piercing member breaks the wall to thereby allow mixture of the mouthwash and saliva with the additional chaotropic agents and/or ethanol 46 within the vessel. The user then agitates the cup 18C (e.g., by shaking it) to contain a lyse and chaotrope sample mixture within the vessel, and then introduces the mixture from the vessel into the inlet 14 of the microfluidic device 12 via, for example, another transfer device, such as a syringe 18A, which may also contain 2-dodecanol (about 50 to about 500 μl (or other long-chain fatty alcohol) 36 for improved assay performance. The 2-dodecanol 36 may sit in a chamber of the syringe 18A such that it is introduced into the device 12 following the saliva-containing mixture.

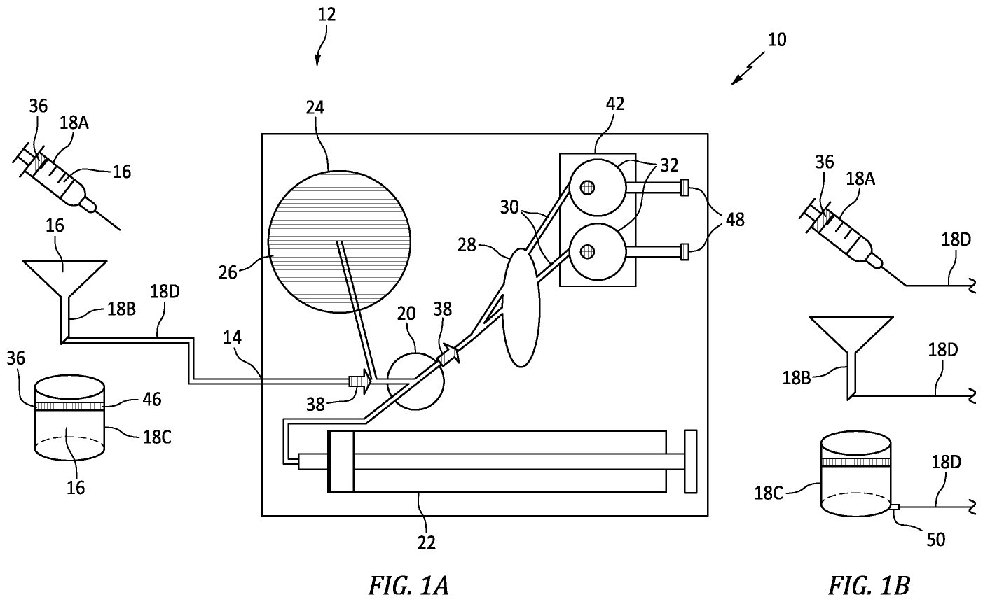

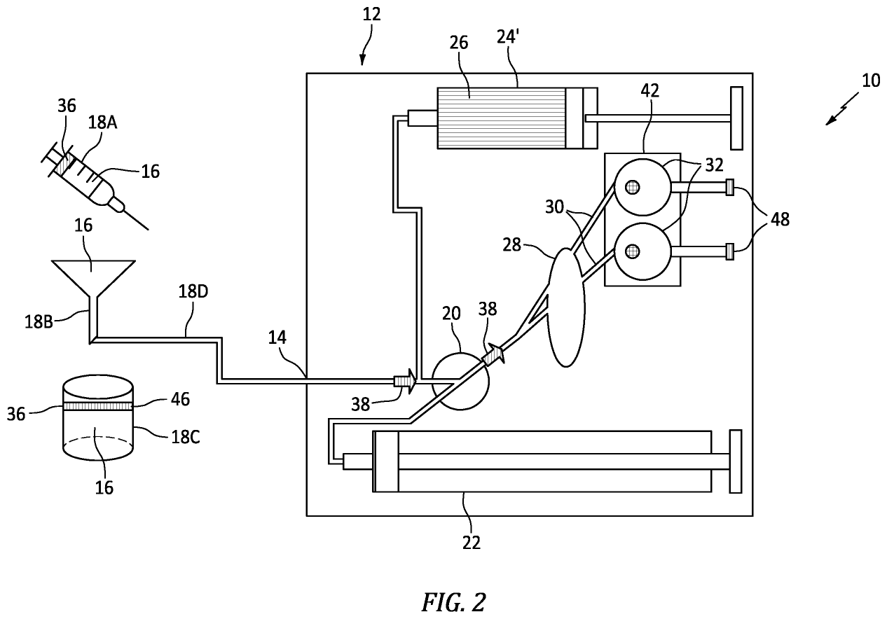

As shown typically in FIGS. 1 and 2, the following are the principal or key components of device 10. The following such components are located outside of the microfluidic device 12:
1) a sample collection formulation or solution 16 (e.g., the mouthwash/nasal spray or wash);
2) a collection device (e.g., a collection cup or vessel 18C, or a swab located at the distal end of a syringe 18A for absorbing the saliva therein, and releasing the saliva when compressed upon actuation of the syringe); and
3) a transfer device (e.g., a syringe 18A or funnel 18B).

The following such components are located on or within the microfluidic device 12:
1) A tube/channel 18D that connects the microfluidic device 12 to the transfer device 18A, 18B and/or 18C;
2) A DNA/RNA (e.g., cfDNA) capture membrane (or solid-state membrane) 20. In the illustrated embodiment of the solid-state membrane, the inlet or inlet side of the membrane is on the top, and the outlet or outlet side of the membrane is on the bottom.
3) A syringe 22 that “pulls” materials from the transfer device/inlet port 14, across the solid-state membrane 20, and into the syringe 22 (defining a waste chamber therein);
4) One-way valves 38, 38 that control the flows of fluids into and across the solid-state membrane 20, and from the solid-state membrane 20 to either the waste chamber in the syringe 22 or to the reservoir 28 for capillary transfer to the reaction chamber(s) 32, 32;
5) A blister mechanism 24, or syringe 24′ or other pump/dispensing device, pre-loaded with an elution buffer 26 (e.g., water). Although standard pull-type syringes 22, 24′ are shown, each syringe may be a reverse pressure syringe, or other type of syringe or like pumping/dispensing device that is currently known, or that later becomes known.
6) A reservoir 28 that pools the elution 26 before loading same into the reaction chambers 32, 32 such that fluids flow from the solid-state membrane 20 into the reservoir 28 and pool within the reservoir before the elution flows into the reaction chamber(s) 32, 32. One advantage of the reservoir 28 is that it can be used to pool the purified RNA/DNA (e.g., cfDNA) to facilitate obtaining a substantially consistent target concentration, including, for example, across multiple reaction chambers;
7) Capillary tubes or conduits 30, 30 that transfer the elution from the reservoir 28 into the reaction chambers 32, 32. One advantage of using capillary tubes is that they provide a capillary fill action into the reaction chamber(s) which, in turn, facilitates bubble management;
8) Reaction chambers 32, 32 pre-loaded with lyophilized amplification reagents (circles lined for the color yellow);
9) A heater 42 for the amplification reaction; and
10) Gas permeable membranes (i.e., gas only valves) 48, 48 at the distal ends of the reaction chambers 32, 32 to allow for air to escape and liquid to fill the respective chambers. As shown in the illustrated embodiment, a plurality of venting lines extend in fluid communication between the reaction chambers 32, 32 and respective gas only valves 48, 48.

The lyophilized amplification reagents contain reagents needed for Loop-Mediated Amplification (LAMP) (e.g., buffers, nucleotides, polymerase enzyme, and target specific oligonucleotides (primers)) and visual detection dye (e.g., Md²⁺, Calcein). Oligonucleotides for the positive control correspond to the beta-actin human housekeeping gene. Oligonucleotides for the target fetal chromosome cfDNA correspond to Y chromosomal nucleotide sequences ranging from 240-500 and/or 5282-5233.

In the operation of the microfluidic system 10, and with reference to FIGS. 1A, 1B and 2, the sample-containing mixture 16 is introduced into the device 12 using options 18A, 18B or 18C and the elution buffer 26 (e.g., water) in encased or contained within the elution blister 24 (FIG. 1A) or the pre-filled elution syringe 24′ (FIG. 2). Three options are illustrated for sample collection and introduction into the device 12. Using option 18A, a collection and transfer syringe is used. If dodecanol 36 is needed, the dodecanol is contained within a sealed section of the syringe 18A and is introduced into the device 12 once the sample is introduced by a lockable or locking mechanism 50 between the syringe and the introduction tube 18D (e.g., a flexible tube). Using option 18B, a funnel is connected to the device 12 via a lockable or locking flexible tube 18D. As can be seen, dodecanol is not used in this particular embodiment. The funnel 18B preferably connects directly to the introduction tube 18D via a locking mechanism 50 of a type known to those of ordinary skill in the pertinent art. Using option 18C, if dodecanol is needed, a chamber within the sample collection vessel 18C contains and releases dodecanol 36 when the vessel is closed. The mixed contents of the sample collection chamber may be “pulled” into the device via the syringe 18A, if desired. The connection between the collection cup 18C and inlet port 14 of the device 12 is a flexible tube 18D having a lockable or locking connection 50 (FIG. 1B) of a type known to those of ordinary skill in the pertinent art.

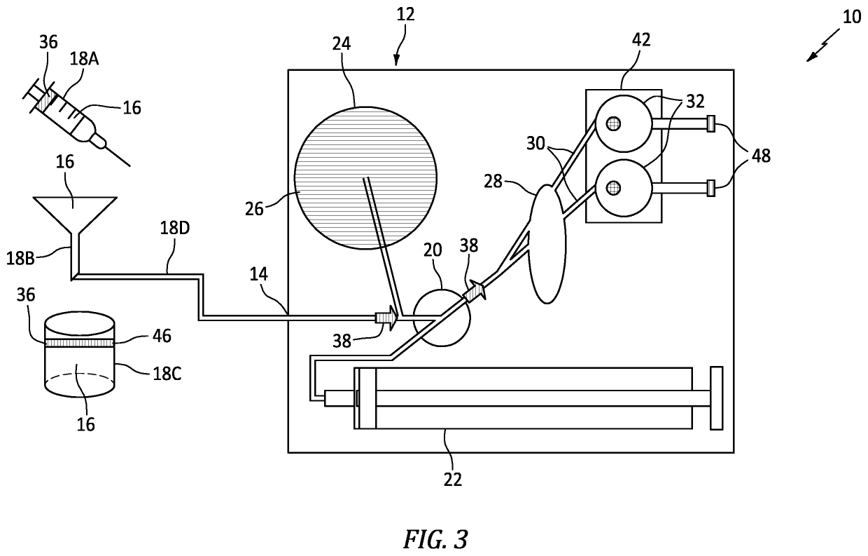

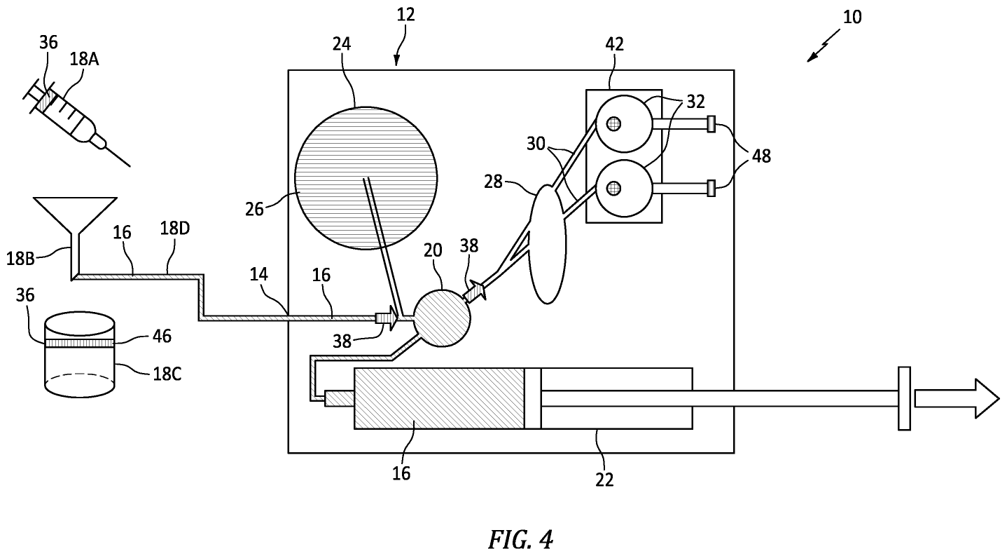

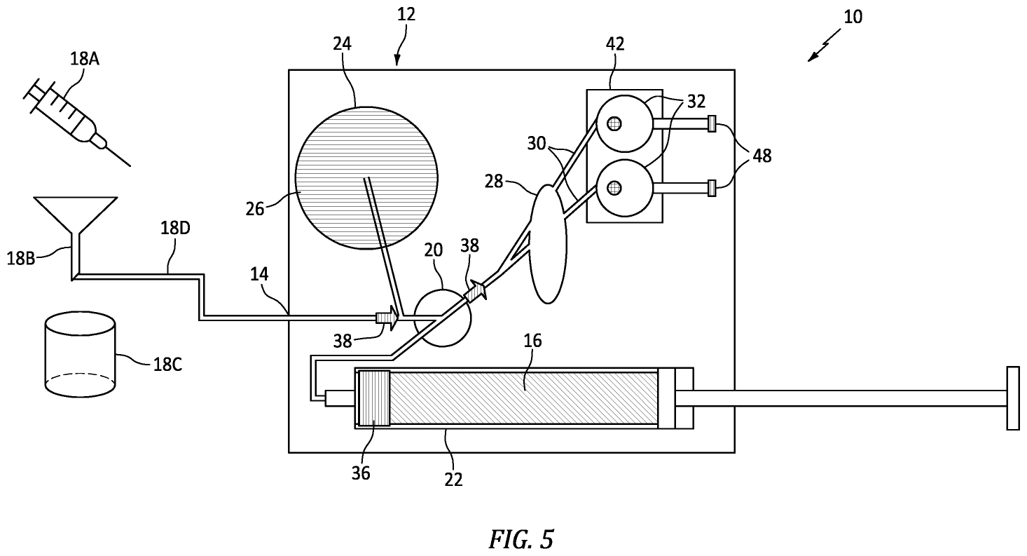

As shown in FIG. 3, a first step of using the device 12 includes the patient or other user collecting a biological sample 16 and introducing the sample mixture 16 into the microfluidic device 12 in accordance with any of options 18A, 18B or 18C described above. As indicated by the arrow in FIG. 4, in the second step, the patient or other user pulls the plunger of the empty syringe 22 which in turn pulls the sample mixture 16 across the capture membrane 20 and into the barrel of the syringe, which acts as waste container. As shown in FIG. 5, in the third step, the patient or other user continues to pull the plunger of the syringe 22 until the plunger hits a stop in the syringe which, in turn, pulls dodecanol 36 across the membrane 20 to remove all residual ethanol. In another embodiment, there is no need for dodecanal, and in the third step, the syringe 22 pulls air across the membrane 20 such that residual air (e.g., residual air in the syringe 18A, funnel 18B, or collection cup 18C) eliminates the ethanol.

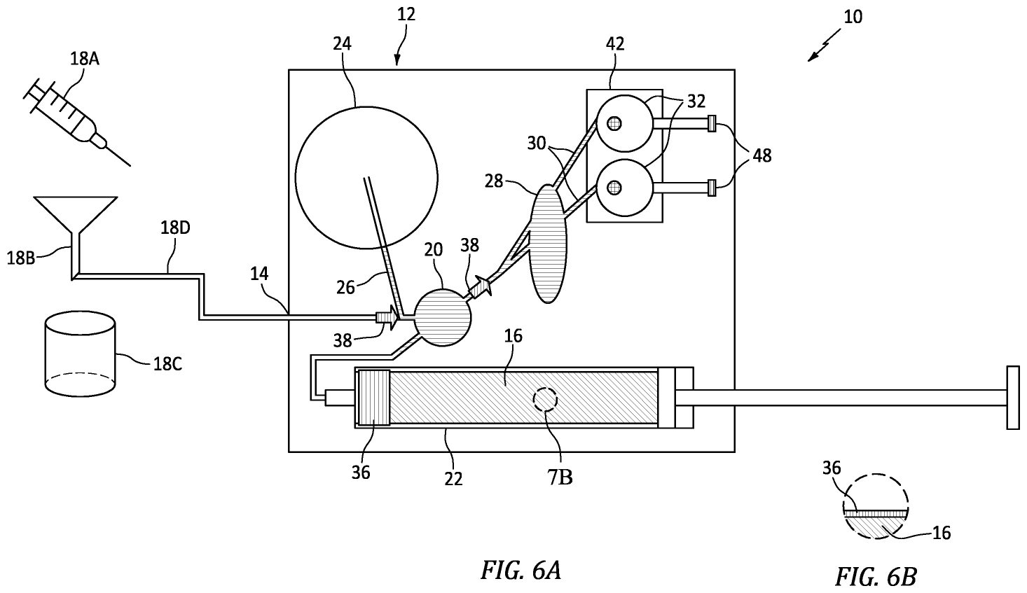

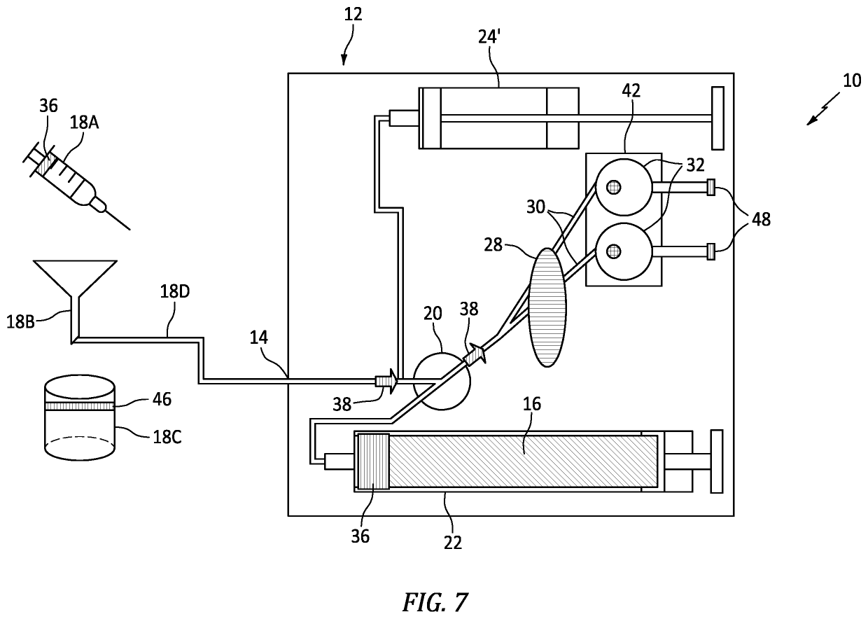

As shown in FIGS. 6A and 6B, in the fourth step, the patient or other user depresses the elution blister 24 which pushes the elution buffer 26 across the membrane 20 to elute all or substantially all DNA and RNA (e.g., cfDNA) off the membrane and into the capture reservoir 28. Fluid flow is controlled by the one-way valves 38, 38 which prevent fluid from flowing backwards from the reaction chambers 32, 32 into the membrane 20 and prevent any material from flowing backwards into the transfer/collection device(s) 18A, 18B, 18C and/or 18D. Once the RNA and DNA (e.g., cfDNA) is in the capture reservoir 28, the reaction chambers 32, 32 are filled via capillary action which dissolves the reaction chemistry pre-stored in the reaction chambers as lyophilized beads. Depression of the elution blister also activates the device heating element 42 for heating the reaction chambers 32, 32 to the required temperature for the reaction. As shown in FIG. 7, in an alternative fourth step of the device 10, the patient or other user depresses the plunger of the elution syringe 24′ which pushes the elution buffer 26 across the membrane 20 to elute all or substantially all DNA and RNA (e.g., cfDNA) off the membrane and into the capture reservoir 28. Fluid flow is controlled by the one-way valves 38, 38 which prevent fluid from flowing backwards from the reaction chambers 32, 32 into the membrane 20 and prevent any material from flowing backwards into the transfer/collection devices 18A, 18B, 18C and/or 18D. Once the RNA and DNA (e.g., cfDNA) is in the capture reservoir 28, the reaction chambers 32, 32 are filled via capillary action which dissolves the reaction chemistry pre-stored in the reaction chambers as lyophilized beads (circles lined for the color yellow). Depression of the elution syringe 24′ also activates the device heater 42.

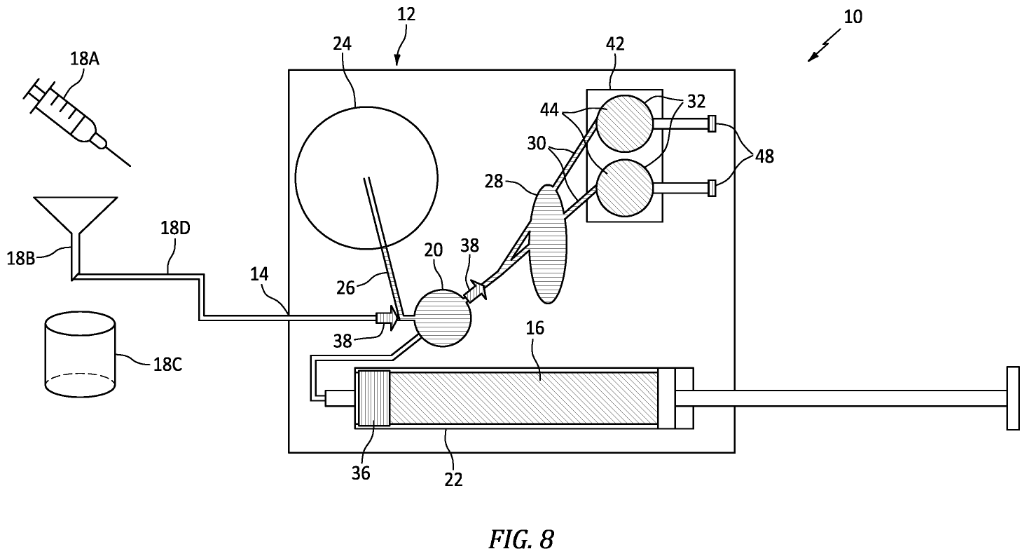

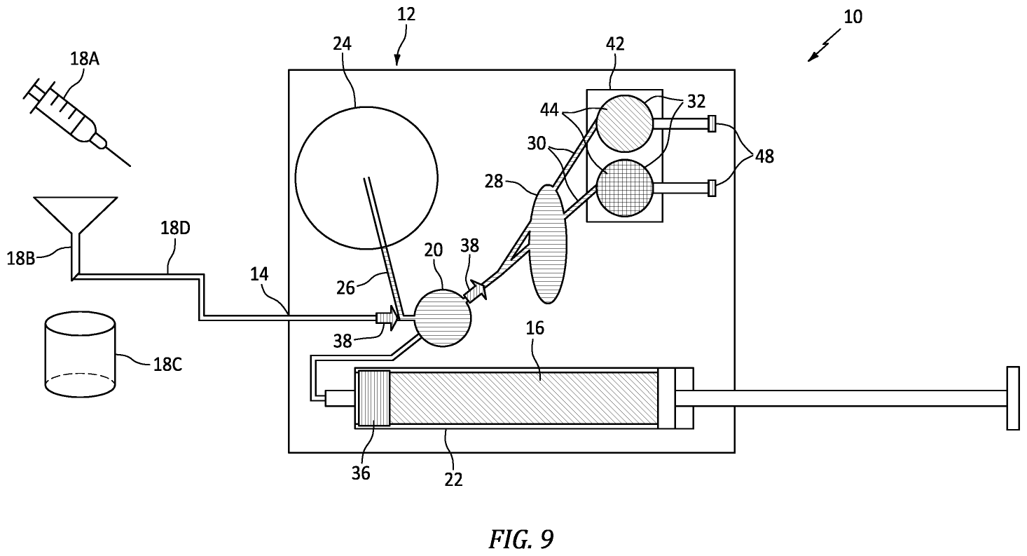

As shown in FIG. 8, in the fifth step of the device 10, heating of the reaction chambers 32, 32 initiates an amplification reaction where the target RNA/DNA (e.g., cfDNA) is amplified, if present. Positive amplification is detected via user visualization (e.g., by color change as shown from blue to green, fluorescence, or a change in turbidity), or by digital image capture via an embedded CCD camera, photo-sensor or an external device such as a smart phone. Although in the illustrated device the heater 42 is an electric heater, a chemical heating or other type of heating mechanism that is currently known, or later becomes known, equally may be employed.

As may be recognized by those of ordinary skill in the pertinent art based on the teachings herein, numerous changes, improvements, modifications, additions, and deletions may be made to the above-described and other embodiments of the present invention without departing from the scope of the invention. For example, the microfluidic devices or components thereof, and the methods of operation or use, or aspects thereof, may be the same as or similar to any of the microfluidic devices or components thereof, and methods or aspects, disclosed in the following co-pending patent applications, which are assigned to the assignee of the present invention and our hereby incorporated by reference in their entireties as part of the present disclosure: (i) U.S. patent application Ser. No. 17/647,828, filed Jan. 12, 2022, entitled “Device And Method For Detecting Nucleic Acids In Biological Samples,” (ii) U.S. patent application Ser. No. 17/941,816, filed Sep. 9, 2022, entitled “Device And Method For Detecting Nucleic Acids In Biological Samples,” and (iii) U.S. patent application Ser. No. 18/176,949, filed Mar. 1, 2023, entitled “Non-Toxic Formulation For Collecting Biological Samples, And Device For Capturing and Eluting Nucleic Acids In The Samples.” In addition, the device may include fewer parts, or additional parts than those illustrated and/or described herein. For example, the device may include only one pump for pumping the sample mixture, and dodecanol and air, any wash solutions and any eluents across one or more solid-state membranes. Alternatively, the device may include multiple pumps for performing such functions. Alternatively, the device may include multiple solid-state membranes or other filtration mechanisms, including membranes mounted in series. Still further, the device may include plural capture reservoirs, or in other cases, the capture reservoir may be eliminated. In other embodiments, the conduit(s) running between the capture reservoir and the reaction chamber(s) and/or negative control chamber(s) need not be capillary or operate by capillary flow action. For example, flow through the conduits may be achieved via pressure differential, such as by the pumping that fills the capture reservoir. In addition, the heating element need not operate in an on/off scenario, but rather may operate by thermo or thermal cycling, such as for PCR or other non-lamp methods/applications. The solid-state membrane also may take the form of any device that is currently known, or that later becomes known for capturing thereon and releasing nucleic acids, RNA and/or DNA (e.g., cfDNA), such as glass beads, including, for example, boro-silicate glass beads. Accordingly, the components of the device(s) and the methods of operating or using the device(s), and the formulations, may take any of numerous different forms or configurations, and may be made of or use any of numerous materials, components or ingredients, that are currently known or later become known, and features or aspects may be added or removed, without departing the from the scope of the invention. This detailed description of embodiments is therefore to be taken in an illustrative as opposed to a limiting sense.

What is claimed is:

1. A device comprising:
a sample port or chamber for receiving therein a sample-containing mixture containing therein a biological sample from a pregnant woman;
a solid-state membrane in fluid communication with the sample port and configured to receive the sample-containing mixture therefrom, and allow the sample-containing mixture to pass across the membrane and capture fetal chromosomal cfDNA in the biological sample on the membrane;
a first pump in fluid communication with at least one of the solid-state membrane or a waste chamber, wherein actuation of the first pump causes the sample-containing mixture to flow across the solid-state membrane and into the waste chamber;
an eluent chamber configured to contain or containing an eluent therein;
an eluent reservoir in fluid communication with the solid-state membrane;
a second pump in fluid communication with at least one of the solid-state membrane or the eluent chamber, wherein actuation of the second pump causes eluent to flow from the eluent chamber across the solid-state membrane and elute fetal chromosomal cfDNA from the solid-state membrane into the eluent reservoir; and
at least one reaction chamber in fluid communication with the eluent reservoir for receiving therefrom the eluent and captured fetal chromosomal cfDNA, and configured for (i) a positive control, and (ii) a detection of Y chromosomal DNA, if any, in the fetal chromosomal cfDNA.

2. A device as defined in claim 1, wherein the first pump is a syringe containing a barrel and a plunger received within the barrel, the barrel defines the waste chamber therein, and movement of the plunger draws or pulls the sample-containing mixture across the solid-state membrane and into waste chamber of the barrel.

3. A device as defined in claim 2, wherein the second pump is movable between a non-actuated position and an actuated position, the eluent chamber includes a frangible or breakable wall that is breakable by movement of the second pump between the non-actuated position and the actuated position to pump eluent from the eluent chamber across the solid-state membrane and into the eluent reservoir.

4. A device as defined in claim 3, wherein the second pump is a plunger, movement of the plunger from the non-actuated position to the actuated position breaks the frangible or breakable wall of the eluent chamber and pushes the eluent across the solid-state membrane and into the eluent reservoir.

5. A device as defined in claim 2, wherein the second pump is a syringe including a barrel and a plunger received within the barrel, and movement of the plunger pushes the eluent across the solid-state membrane and into the eluent reservoir.

6. A device as defined in claim 1, further comprising a first one-way valve in fluid communication between the sample port or chamber and the solid-state membrane and configured to allow the sample-containing mixture to flow in the direction from the sample port or chamber to the solid-state membrane.

7. A device as defined in claim 1, further comprising a second one-way valve in fluid communication between the solid-state membrane and the eluent reservoir and configured to allow fluid flow in the direction from the solid-state membrane into the eluent reservoir.

8. A device as defined in claim 1, comprising a plurality of reaction chambers in fluid communication with the eluent reservoir for receiving therefrom the eluent and captured nucleic acids, including a first reaction chamber configured for a positive control, and a second reaction chamber configured for the detection of Y chromosomal DNA, if any, in the fetal chromosomal cfDNA.

9. A device as defined in claim 8, further comprising at least one capillary conduit in fluid communication between the eluent reservoir and at least one reaction chamber, wherein the capillary conduit is configured to allow the eluent with captured nucleic acid to flow by capillary action through the capillary conduit and into the reaction chamber.

10. A device as defined in claim 9, further comprising a plurality of capillary conduits in fluid communication between the eluent reservoir and the reaction chambers, wherein each capillary conduit is configured to allow the eluent with captured nucleic acid to flow by capillary action through the capillary conduit and into a respective reaction chamber.

11. A device as defined in claim 1, further comprising a valve in fluid communication between the reaction chamber and an ambient atmosphere, where the valve allows gas to flow from the reaction chamber into the ambient atmosphere, but prevents liquid flow therethrough.

12. A device as defined in claim 1, further comprising a heating element in thermal communication with the reaction chamber, wherein the heating element defines a first condition where the heating element heats the reaction chamber to an incubation temperature, and a second condition where the heating element does not heat the reaction chamber to the incubation temperature, and the heating element is configured to transition from the second condition to the first condition upon or following actuation of the second pump.

13. A device as defined in claim 1, further comprising an optical sensor configured to measure at least one of emission wavelength or intensity within the reaction chamber to (a) detect at least one of (i) a positive detection of Y chromosomal DNA in the fetal chromosomal cfDNA or (ii) a negative detection of Y chromosomal DNA in the fetal chromosomal cfDNA, and (b) transmit a signal to a user interface indicative thereof.

14. A device comprising:
first means for receiving therein a sample-containing mixture containing therein a biological sample from a pregnant woman;
second means in fluid communication with the first means for receiving the sample-containing mixture therefrom, for allowing the sample-containing mixture to pass across the second means, and for capturing fetal chromosomal cfDNA in the biological sample on the membrane;
third means for receiving and holding the sample-containing mixture after passing across the second means;
fourth means in fluid communication with at least one of the second means or the third means for pumping the sample-containing mixture across the second means and into the third means;
fifth means for containing an eluent therein and for allowing the eluent to flow across the second means after the sample-containing mixture passes across the second means, and for removing from the second means fetal chromosomal cfDNA from the biological sample with the eluent;
sixth means in fluid communication with the second means for receiving and collecting the eluent with captured fetal chromosomal cfDNA from the biological sample therein;
seventh means in fluid communication with at least one of the second means or the fifth means for pumping the eluent from the fifth means across the second means and eluting fetal chromosomal cfDNA from the second means into the sixth means; and
eighth means in fluid communication with the sixth means for receiving therefrom the eluent and captured fetal chromosomal cfDNA and for detecting Y chromosomal DNA, if any, in the fetal chromosomal cfDNA.

15. A device as defined in claim 13, wherein the first means is a sample port or chamber, the second means is a solid-state membrane, the third means is a waste chamber, the fourth means is a pump, the fifth means is an eluent chamber, the sixth means is an eluent reservoir, the seventh means is a pump, and the eighth is a reaction chamber configured for a positive control, and another reaction chamber configured for the detection of Y chromosomal DNA, if any, in the fetal chromosomal cfDNA.

16. A formulation for collecting a biological sample of saliva or fluid from a pregnant woman and capturing nucleic acids in the collected biological sample on a solid-state membrane, including chromosomal cfDNA and a Y chromosome nucleotide sequence, if any, in the collected biological sample, comprising: one or more non-toxic chaotropic agents; ethanol; and/or coloring and/or flavoring agents, wherein the formulation is receivable within an oral cavity or other cavity of the pregnant woman to collect the biological sample of saliva or fluid therefrom, the one or more non-toxic chaotropic agents at least one of lyse the cells of the biological sample, or bind or facilitate binding of the chromosomal cfDNA, and a Y chromosome nucleotide sequence, if any, in the cells of the biological sample, to the solid-state membrane.

17. A formulation as defined in claim 15, comprising about 0.1% to about 40% w/v non-toxic chaotropic agents and about 5% to about 30% w/v ethanol.

18. A formulation as defined in claim 16, wherein the non-toxic chaotropic agents are selected from the group including the following individually or in any combination thereof: (i) about 5% to about 30% w/v urea; about 0.1% to about 3% w/v sodium lauryl sulfate; and about 2% to about 40% w/v ammonium trichloroacetate.

19. A formulation as defined in claim 15, in combination with a long-chain fatty alcohol wash configured to flow over the solid-state membrane following the formulation to substantially eliminate any residual ethanol of the formulation on the solid-state membrane.

20. A combination as defined in claim 18, further comprising a transfer device containing in a first portion thereof the formulation and containing in a second portion thereof the long-chain fatty alcohol wash, wherein the first portion thereof is receivable within an oral cavity or other cavity of the pregnant woman for collecting saliva or fluid, and the transfer device is configured to transfer or dispense the formulation and collected biological sample to the solid-state membrane and then transfer or dispense the long-chain fatty alcohol wash to the solid-state membrane.

21. A combination as defined in claim 19, wherein the transfer device is a syringe comprising on a distal end thereof a gauze or other absorbent material that is receivable in the oral cavity or other cavity of the pregnant woman and configured to absorb the saliva or other fluid therein, wherein the syringe defines a first chamber or portion of a chamber in fluid communication with the gauze or other absorbent material and containing therein the formulation, and a second chamber or portion of a chamber located on an opposite side of the first chamber or portion thereof relative to the gauze or other absorbent material and containing therein the long-chain fatty alcohol wash, wherein actuation of the syringe causes the formulation to flow through the gauze or other absorbent material and collect therein the biological sample and to dispense from the syringe a mixture of the formulation and biological sample, and further actuation of the syringe causes the long-chain fatty alcohol wash to be dispensed from the syringe following dispensing of the formulation and biological sample mixture.

22. A combination as defined in claim 20, wherein the syringe includes a barrel defining the first and second chambers and including a frangible or breakable wall separating the first and second chambers, and the syringe further comprises a plunger received within the barrel, whereupon actuation of the plunger breaks the frangible or breakable wall and dispenses the formulation and then the long-chain fatty alcohol wash from the syringe.
